(19) United States
(12) Patent Application Publication
(10) Pub. No.: US 2023/0159427 A1
(43) Pub. Date: May 25, 2023

(54) METHOD FOR PRODUCING CATALYST FOR DEHYDROGENATION OF 3-HYDROXYPROPIONIC ACID, CATALYST FOR DEHYDROGENATION OF 3-HYDROXYPROPIONIC ACID, AND METHOD FOR PRODUCING ACRYLIC ACID USING SAME

(71) Applicant: LG CHEM, LTD., Seoul (KR)

(72) Inventors: Jaeyoung KIM, Daejeon (KR); Jungup BANG, Daejeon (KR); Jae Soon CHOI, Daejeon (KR)

(21) Appl. No.: 17/915,054

(22) PCT Filed: Oct. 14, 2021
(86) PCT No.: PCT/KR2021/014200

§ 371 Date: Sep. 27, 2022

(30) Foreign Application Priority Data
Nov. 27, 2020 (KR) 10-2020-0163037

(51) Int. Cl. C07C 51/377; B01J 27/18; B01J 37/08; B01J 37/04

(52) U.S. Cl. CPC C07C 51/377; B01J 27/1806; B01J 37/08; B01J 37/04

(57) ABSTRACT

A method for preparing a catalyst for a dehydration reaction of 3-hydroxypropionic acid, the catalyst for dehydration reaction of 3-hydroxypropionic acid, and a method for producing acrylic acid using the same.

### CROSS-REFERENCE TO RELATED APPLICATIONS

This application is a US national phase of international Application No. PCT/KR2021/014200 filed Oct. 14, 2021, and claims priority to and the benefits of Korean Patent Application No. 10-2020-0163037, filed on Nov. 27, 2020, the entire contents of which are incorporated herein by reference.

### TECHNICAL FIELD

The present specification relates to a method for preparing a catalyst for dehydration reaction of 3-hydroxypropionic acid, the catalyst for dehydration reaction of 3-hydroxypropionic acid, and a method for producing acrylic acid using the same.

### BACKGROUND ART

3-Hydroxypropionic acid (3-HP) is a platform compound capable of being converted into various substances and may be obtained from glucose or glycerol through a biological conversion process.

3-hydroxypropionic acid (3-HP) may be converted to acrylic acid through a dehydration reaction, wherein the dehydration reaction is carried out in the presence of: solid acid catalysts such as SiO₂, TiO₂, Al₂O₃, and zeolite; base catalysts such as ammonia, polyvinylpyrrolidine, metal hydroxide, and Zr(OH)₄; or metal catalysts such as MgSO₄, Al₂(SO₄)₃, K₂SO₄, AlPO₄, and Zr(SO₄)₂, and the yield of acrylic acid produced is a level of 60% to 90%.

Relevant Patent: KR 10-2012-0025888 A

### SUMMARY

The present disclosure relates to a method for preparing a catalyst for dehydration reaction of 3-hydroxypropionic acid, the catalyst for dehydration reaction of 3-hydroxypropionic acid, and a method for producing acrylic acid using the same.

An embodiment of the present disclosure provides a method for preparing a catalyst for dehydration reaction of 3-hydroxypropionic acid, including hydroxyapatite (HAP) and calcium pyrophosphate, the method comprising the steps of: preparing an apatite cake by dropping a first phosphate solution into a first calcium salt solution; preparing a calcium pyrophosphate (CaPP) cake by dropping a second phosphate solution into a second calcium salt solution; preparing a calcium phosphate cake by mixing the apatite cake and the calcium pyrophosphate cake; and firing the calcium phosphate cake.

An embodiment of the present disclosure provides a catalyst for dehydration reaction of 3-hydroxypropionic acid, which includes a hydroxyapatite phase and a calcium pyrophosphate phase in which the hydroxyapatite phase and the calcium pyrophosphate phase of the catalyst have a weight ratio of 10:90 to 80:20.

Furthermore, an embodiment of the present disclosure provides a method for producing acrylic acid, comprising the step of producing acrylic acid by performing dehydration reaction of 3-hydroxypropionic acid using the catalyst.

The catalyst prepared by the method for preparing a catalyst for dehydration reaction of 3-hydroxypropionic acid according to an embodiment of the present disclosure exhibits a high acrylic acid yield when used in a process for producing acrylic acid by performing dehydration reaction of 3-hydroxypropionic acid.

### DETAILED DESCRIPTION

In the present specification, when a part ‘includes’ a certain component, it means that other components may be further included, rather than excluding other components, unless there is a particular contrary description.

The term ‘phase’ of a substance in the present specification means a state in which certain substances are gathered to form a system or group having uniform physical and chemical properties from a macroscopic point of view. The existence of such a phase can be confirmed through XRD analysis under specific voltage, current, scan speed and scan step conditions using Bruker's D4 endeavor X-ray diffractometer. That is, a peak occurring within a specific 2θ (theta) range may be observed and confirmed by the result of XRD analysis.

Hereinafter, the present disclosure will be described in more detail.

An embodiment of the present disclosure provides a method for preparing a catalyst for dehydration reaction of 3-hydroxypropionic acid, including hydroxyapatite (HAP) and calcium pyrophosphate, the method comprising the steps of: preparing an apatite cake by dropping a first phosphate solution into a first calcium salt solution; preparing a calcium pyrophosphate (CaPP) cake by dropping a second phosphate solution into a second calcium salt solution; mixing the apatite cake and the calcium pyrophosphate cake to form a calcium phosphate cake; and firing the calcium phosphate cake.

When the catalyst includes only hydroxyapatite (HAP) or calcium pyrophosphate, there has been a problem in that the yield of acrylic acid in the 3-hydroxypropionic acid dehydration reaction is lowered and the processability is deteriorated due to a short lifespan.

An advantage of a catalyst comprising both hydroxyapatite and calcium pyrophosphate is that the yield of the dehydration reaction is improved, and the processability is improved due to a long lifespan. When the catalyst comprises hydroxyapatite and calcium pyrophosphate, it may be defined as having a mixed phase of hydroxyapatite and calcium pyrophosphate. The mixed phase means that hydroxyapatite and calcium pyrophosphate are simply mixed.

The apatite cake and the calcium pyrophosphate cake are prepared by reacting phosphate solutions with calcium salt solutions, and the present disclosure has been completed by studying that the mixed phase is easily formed when preparing each cake by dropping the phosphate solution into the calcium salt solution, which is used as the mother liquid, instead of dropping the calcium salt solution into the phosphate solution as in a conventional process.

Specifically, a method for preparing a catalyst according to an embodiment of the present disclosure comprises the steps of: preparing an apatite cake by dropping a first phosphate solution into a calcium salt solution; and preparing a calcium pyrophosphate cake by dropping a second phosphate solution into the calcium salt solution.

In the present specification, dropping means dropping a solution in the form of drops, and the dropping amount may be adjusted by adjusting the dropping rate (unit: volume/time, e.g., mL/min) and the dropping time. A conventional method may be used for the dropping.

In an embodiment of the present disclosure, the step of preparing the apatite cake by dropping the first phosphate solution into the first calcium salt solution comprises a step of dropping the first phosphate solution into a bath containing the first calcium salt solution, and the step of preparing the calcium pyrophosphate cake by dropping the second phosphate solution into the second calcium salt solution comprises a step of dropping the second phosphate solution into a bath containing the second calcium salt solution.

In an embodiment of the present disclosure, the step of preparing the apatite cake by dropping the first phosphate solution into the first calcium salt solution comprises a step of dropping the first phosphate solution into a bath containing the first calcium salt solution. Specifically, after preparing the bath containing the first calcium salt solution, the first phosphate solution may be slowly dropped into the bath.

In an embodiment of the present disclosure, the step of preparing the calcium pyrophosphate cake by dropping the phosphate solution into the second calcium salt solution comprises a step of dropping the second phosphate solution into the bath containing the second calcium salt solution. Specifically, after preparing the bath containing the second calcium salt solution, the second phosphate solution may be slowly dropped into the bath.

The dropping rate may be 0.1 mL/min or more and 10 mL/min or less, or 1 mL/min or more and 10 mL/min or less. If of the dropping rate is lower than the above range, the reaction is slow, which deteriorates processability, and if the dropping rate is exceeded, a reaction may occur rapidly and precipitates may be generated. Thus, it is preferable to adjust the reaction within the above range.

The step of preparing a calcium phosphate salt by dropping the phosphate solution into the calcium salt solution is different from a conventional method of preparing a calcium phosphate salt by dropping a calcium salt solution into a phosphate solution. When dropping the calcium salt solution into the phosphate solution as in the conventional method, there has been a problem in that a sodium pyrophosphate (Na₂CaP₂O₇) hydrate is produced and a calcium pyrophosphate phase is not well formed after heat treatment so that the mixed phase of hydroxyapatite (HAP) and calcium pyrophosphate is not formed well.

Meanwhile, as in an embodiment of the present disclosure, when preparing the calcium phosphate salt by dropping the phosphate solution into the calcium salt solution, the mixed phase of hydroxyapatite and calcium pyrophosphate may be easily formed due to the formation of a calcium pyrophosphate (Ca₂P₂O₇) hydrate.

The existence of a mixed phase may be confirmed using XRD (X-ray diffraction) analysis of the prepared catalyst. Bruker's D4 endeavor X-ray diffractometer can be used for the XRD analysis, and the existence of the mixed phase may be measured by observing a peak occurring within a 2θ (theta) range of 10 to 60 degrees (°) under a CuKa lamp, 40 kV voltage, 40 mA current, 0.8 deg/min scan speed, and 0.015 scan step conditions.

Specifically, the existence of a mixed phase can be confirmed by confirming that the peak corresponding to the hydroxyapatite phase at 2θ (theta) angles of 25.9 degrees (°) and 31.7 degrees (°) and the peak corresponding to the calcium pyrophosphate (CaPP) phase at 2θ (theta) angles of 26.7 degrees (°) and 30.5 degrees (°) appear at the same time.

In an embodiment of the present specification, the apatite cake and the calcium pyrophosphate cake in the step of preparing the calcium phosphate cake by mixing the apatite cake and the calcium pyrophosphate cake may have a weight ratio of 10:90 to 80:20, 15:85 to 70:30, 18:82 to 55:45, or 20:80 to 50:50.

Further, in an embodiment of the present specification, the apatite cake may have a weight ratio smaller than that of the calcium pyrophosphate cake in the step of preparing the calcium phosphate cake by mixing the apatite cake and the calcium pyrophosphate cake. When the apatite cake has a weight ratio smaller than that of the calcium pyrophosphate cake in the step of preparing the calcium phosphate cake by mixing the apatite cake and the calcium pyrophosphate cake, the yield of acrylic acid may be further increased when producing acrylic acid using a catalyst for dehydration reaction of hydroxypropionic acid prepared using the calcium phosphate cake.

In an embodiment of the present specification, the first calcium salt solution and the second calcium salt solution may each have a calcium salt concentration of 0.1M or more and 5M or less.

In an embodiment of the present specification, the first calcium salt solution and the second calcium salt solution may each have a calcium salt concentration of 0.1M or more and 5M or less, preferably 0.3M or more and 2M or less, and more preferably 1M or more and 1.5M or less. When the above range is exceeded, the particles are hardened during stirring due to gelation of calcium pyrophosphate, and when it falls short of the above range, the catalyst productivity is low, which are both problematic.

In an embodiment of the present specification, the first phosphate solution and the second phosphate solution may each have a phosphate concentration of 0.1M or more and 5M or less. In an embodiment of the present specification, the first phosphate solution and the second phosphate solution may each have a phosphate concentration of 0.1M or more and 5M or less, preferably 0.3M or more and 2M or less, and more preferably 0.5M or more and 1M or less. When the above range is exceeded, the particles are hardened during stirring due to gelation of calcium pyrophosphate, and when it falls short of the above range, the catalyst productivity is low, which are both problematic.

In an embodiment of the present specification, the dropping of the first phosphate solution and the dropping of the second phosphate solution into the respective calcium salt solutions may be performed for 25 seconds or more and 1 hour or less, and preferably 30 seconds or more and 1 hour or less.

In an embodiment of the present specification, the first phosphate may be one or more selected from the group consisting of Li₃PO₄, Na₃PO₄, and K₃PO₄, or hydrates thereof.

In an embodiment of the present specification, the second phosphate may be one or more selected from the group consisting of pyrophosphoric acid (H₄P₂O₇), Li₄P₂O₇, Na₄P₂O₇, and K₄P₂O₇, or hydrates thereof.

In an embodiment of the present specification, the first calcium salt solution and the second calcium salt solution may each contain one or more calcium salts selected from the group consisting of calcium chloride, calcium nitrate, calcium sulfate, and calcium acetate, or hydrates thereof.

In an embodiment of the present specification, the step of preparing the calcium phosphate cake by mixing the phosphate solution and the calcium salt solution may comprise a step of performing stirring at a temperature of 20° C. to 60° C. for 1 hour to 48 hours.

In an embodiment of the present specification, the step of preparing the calcium phosphate cake by mixing the apatite cake and the calcium pyrophosphate cake may comprise the steps of: preparing a calcium phosphate slurry by mixing the apatite cake and the calcium pyrophosphate cake; and filtering, washing, and drying the calcium phosphate slurry to prepare a calcium phosphate cake.

In an embodiment of the present specification, the step of preparing the calcium phosphate cake by mixing the apatite cake and the calcium pyrophosphate cake may further comprise a step of pulverizing the calcium phosphate cake.

The step of pulverizing the calcium phosphate cake may comprise a step of pulverizing the calcium phosphate cake into a powder having an average particle size (D50) of 5 μm to 100 μm. The average particle size (D50) means a particle size when the cumulative percentage in the average particle size distribution reaches 50%. Horiba's Laser Particle Size Analyzer LA-950 equipment may be used to measure the average particle size (D50). To measure the (D50) value, a dispersion is prepared by diluting a solution containing the calcium phosphate powder with water, injecting the dispersion into a Laser Particle Size Analyzer, and measuring the (D50) value at a temperature range of 15° C. to 35° C.

In an embodiment of the present specification, the step of filtering, washing, and drying the calcium phosphate slurry may be performed at 80° C. to 120° C. for 5 hours to 48 hours.

In an embodiment of the present specification, the method may further comprise a step of molding the calcium phosphate cake or powder into calcium phosphate pellets.

In an embodiment of the present specification, the step of firing the calcium phosphate cake may be performed at a temperature of 300° C. to 700° C. The temperature condition may be adjusted to 400° C. to 600° C., or 450° C. to 550° C. so that the firing can be sufficiently performed.

In an embodiment of the present specification, the step of firing the calcium phosphate cake may be performed for 1 hour to 24 hours.

An embodiment of the present disclosure provides a catalyst for dehydration reaction of 3-hydroxypropionic acid including a hydroxyapatite phase and a calcium pyrophosphate phase. The catalyst may be prepared by the above-described preparation method.

In an embodiment of the present specification, the hydroxyapatite phase and the calcium pyrophosphate phase of the catalyst for dehydration reaction may have a weight ratio of 10:90 to 80:20.

In an embodiment of the present specification, the hydroxyapatite phase and the calcium pyrophosphate phase of the catalyst for dehydration reaction may have a weight ratio of 10:90 to 80:20, 15:85 to 70:30, 18:82 to 55:45, or 20:80 to 50:50. The weight ratio of the hydroxyapatite phase and the calcium pyrophosphate phase is the same as the weight ratio of the apatite cake and the calcium pyrophosphate cake in the step of preparing the calcium phosphate cake by mixing the apatite cake and the calcium pyrophosphate cake.

Further, in an embodiment of the present specification, the hydroxyapatite phase of the catalyst for dehydration reaction may have a weight ratio smaller than that of the calcium pyrophosphate phase.

When the weight ratio of the hydroxyapatite phase of the catalyst for dehydration reaction is smaller than that of the calcium pyrophosphate phase, the yield of acrylic acid may be further increased when producing acrylic acid using the catalyst for dehydration reaction.

In an embodiment of the present disclosure, calcium pyrophosphate may be p-calcium pyrophosphate (β-Capp). Further, an embodiment of the present disclosure provides a method for producing acrylic acid, comprising the step of producing acrylic acid by performing dehydration reaction of 3-hydroxypropionic acid using the catalyst described herein.

3-hydroxypropionic acid may be prepared by converting glucose or glycerol. For example, it can be prepared by preparing a product containing allyl alcohol from a reactant containing glycerol and carboxylic acid, injecting a heterogeneous catalyst and a basic solution into the product, and oxidizing the product. A specific preparation method is as described in Patent Document KR 10-2015-0006349 A.

The step of producing acrylic acid by performing dehydration reaction of 3-hydroxypropionic acid is performed as described in the following reaction:

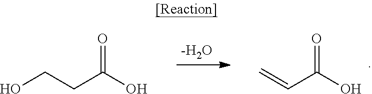

The dehydration reaction may be carried out in any one reactor selected from the group consisting of a batch reactor, a semi-batch reactor, a continuous stirring tank reactor, a plug flow reactor, a fixed bed reactor, and a fluidized bed reactor, which are provided with the catalyst for dehydration reaction of 3-hydroxypropionic acid, or a mixed reactor to which two or more of the above reactors are connected.

The dehydration reaction may be carried out at a temperature of 70° C. to 400° C., preferably 100° C. to 300° C., and more preferably 70° C. to 280° C.

The dehydration reaction may be operated under reduced pressure, normal pressure, and high pressure. Preferably, a vacuum distillation method may be used. During vacuum distillation, the pressure may be controlled to 5 mbar to 50 mbar, 10 mbar to 40 mbar, or 15 mbar to 30 mbar. If the pressure is less than 5 mbar, organic solvents other than acrylic acid are also recovered, and thus, it may be difficult to recover high-purity acrylic acid. If the pressure exceeds 50 mbar, the dehydration reaction to acrylic acid may not occur, or the recovery of acrylic acid may be difficult.

Hereinafter, the present disclosure will be described in more detail through Examples. However, the following Examples are means for explaining the present disclosure, and the right scope of the present disclosure is not limited to the matters described in Examples.

<Preparation of Catalyst>

### (1) Example 1

A first phosphate solution A in which 23 g (=0.75 M) of sodium phosphate (Na₃PO₄12H₂O) had been dissolved in 80 mL of distilled water, and a calcium salt solution B (molar concentration: 1.19 M) in which 14 g of a calcium chloride hydrate (CaCl₂2H₂O) had been dissolved in 80 mL of distilled water were prepared.

A bath was prepared by dropping the calcium salt solution B into 40 ml of distilled water of room temperature at a rate of 3 ml/min for 30 seconds. The first phosphate solution was dropped into the bath at a rate of 3 ml/min for 30 seconds. At this time, particles may be formed under conditions in which the concentration of calcium salt in the mother liquid of the bath is high during dropping.

A Ca₅(PO₄)₃OH cake was prepared by filtering, filtering, and washing a slurry formed while the solution was mixed.

A second phosphate solution C in which 23 g (=0.21 M) of sodium pyrophosphate (Na₂P₂O₇) had been dissolved in 180 mL of distilled water, and a calcium salt solution D (molar concentration: 1.02 M) in which 27 g of a calcium chloride hydrate (CaCl₂2H₂O) had been dissolved in 180 mL of distilled water were prepared.

A bath was prepared by dropping the calcium salt solution D into 40 ml of distilled water of room temperature at a rate of 3 ml/min for 30 seconds. The second phosphate solution was dropped into the bath at a rate of 3 ml/min.

A β-calcium pyrophosphate cake was prepared by filtering, and washing a slurry formed while the solution was mixed.

The prepared apatite (Ca₅(PO₄)₃OH) cake and β-calcium pyrophosphate cake were mixed in 1 L of water at a weight ratio of 20:80, and stirred at room temperature for about 2 hours. Thereafter, the process of filtering the stirred solution was repeated three times. Thereafter, a cake obtained through the washing process was dried and fired at 500° C. to prepare a mixed-phase catalyst having a weight ratio of the hydroxyapatite phase to the calcium pyrophosphate phase of 20:80.

### (2) Example 2

A mixed-phase catalyst having a weight ratio of the hydroxyapatite phase and the calcium pyrophosphate phase of 70:30 was prepared in the same manner as in Example 1 except that the prepared apatite (Ca₅(PO₄)₃OH) cake and β-calcium pyrophosphate cake were mixed in 1 L of water at a weight ratio of 70:30.

### (3) Example 3

A mixed-phase catalyst having a weight ratio of the hydroxyapatite phase to the calcium pyrophosphate phase of 15:85 was prepared in the same manner as in Example 1 except that the prepared apatite (Ca₅(PO₄)₃OH) cake and β-calcium pyrophosphate cake were mixed in 1 L of water at a weight ratio of 15:85.

### (4) Comparative Example 1

A first phosphate solution A in which 23 g of sodium phosphate (Na₃PO₄12H₂O) had been dissolved in 80 mL of distilled water and a calcium salt solution B in which 14 g of a calcium chloride hydrate (CaCl₂2H₂O) had been dissolved in 80 mL of distilled water were prepared.

A bath was prepared by dropping the calcium salt solution B into 40 ml of distilled water of room temperature at a rate of 3 ml/min for 30 seconds. The first phosphate solution was dropped into the bath at a rate of 3 ml/min.

A Ca₅(PO₄)₃OH cake was prepared by filtering, and washing a slurry formed while the solution was mixed.

After mixing the prepared Ca₅(PO₄)₃OH cake with 1 L of water, the process of filtering the mixed solution was repeated three times. Thereafter, a cake obtained through the washing process was dried and fired at 500° C. to prepare a HAP single-phase catalyst.

### (5) Comparative Example 2

A second phosphate solution C in which 23 g (=0.21 M) of sodium pyrophosphate (Na₂P₂O₇) had been dissolved in 180 mL of distilled water and a calcium salt solution D (molar concentration: 1.02 M) in which 27 g of a calcium chloride hydrate (CaCl₂2H₂O) had been dissolved in 180 mL of distilled water were prepared.

A bath was prepared by dropping the calcium salt solution D into 40 ml of distilled water of room temperature at a rate of 3 ml/min for 30 seconds. The second phosphate solution was dropped into the bath at a rate of 3 ml/min.

A β-calcium pyrophosphate cake was prepared by filtering, filtering, and washing a slurry formed while the solution was mixed.

After mixing the prepared β-calcium pyrophosphate cake with 1 L of water, the process of filtering water by filtering the mixed solution was repeated three times. Thereafter, a cake obtained through the washing process was dried and fired at 500° C. to prepare a β-calcium pyrophosphate single-phase catalyst.

### (6) Comparative Example 3

A titanium dioxide catalyst having an anatase phase (TiO₂, manufactured by Thermo Fisher Scientific) was prepared.

### (7) Comparative Example 4

A silica gel catalyst (silica gel, manufactured by Aladdin) was prepared.

### <Experimental Example> Dehydration Reaction of 3-Hydroxypropionic Acid

After filling 0.4 g of the catalyst prepared in Example 1 above in a quartz reaction tube having a diameter of ½ inch, the reaction tube was connected and fastened to a reactor furnace.

The temperature was raised to a reaction temperature of 280° C. (temperature increase rate: about 12.5° C./min) while purging with a nitrogen carrier gas (N₂) in the reaction tube at a rate of 25 sccm.

After reaching the reaction temperature, an aqueous 3-hydroxypropionic acid solution (concentration: 20% by weight) was injected into an upper end portion of the reactor at 1.5 mL/h using a syringe pump.

The injected 3-hydroxypropionic acid was vaporized within the reaction tube and was converted to acrylic acid through dehydration reaction in the presence of a catalyst. After that, acrylic acid condensed in a liquid phase was obtained in a sample trap (maintained at 3° C.) at a lower end portion of the reaction tube (Experimental Example 1). High-performance Liquid Chromatography (HPLC) and Gas Chromatography Flame Ionization Detector (GC-FID) were used to quantify production of acrylic acid, and production and consumption amount of 3-hydroxypropionic acid in mole numbers.

The yield of acrylic acid obtained by Experimental Example 1 was 94.9%.

In addition, acrylic acid was produced by performing dehydration of 3-hydroxypropionic acid in the same manner as in producing acrylic acid using the catalyst of Example 1 except that the experimental conditions of the catalysts of Examples 2 and 3 and Comparative Examples 1 to 4 prepared above such as catalyst loading amount, reaction temperature, carrier gas, and carrier gas flow amount were changed as shown in Table 1 below (Experimental Examples 2 to 10).

The yields of acrylic acid obtained by Experimental Examples 2 to 10 are as shown in Table 1 below.

The yields of acrylic acid were calculated through Equations 1 to 3 below:

3-hydroxypropionic acid conversion rate (%)=100×(mole number of 3-hydroxypropionic acid before reaction−mole number of 3-hydroxypropionic acid after reaction)/(mole number of 3-hydroxypropionic acid before reaction); [Equation 1]

Acrylic acid selectivity (%)=100×(mole number of acrylic acid produced)/(mole number of 3-hydroxypropionic acid reacted); [Equation 2]

Acrylic acid yield (%)=(3-hydroxypropionic acid conversion rate X acrylic acid selectivity)/100. [Equation 3]

TABLE 1

| | | | | | Carrier | Acrylic |
|---|---|---|---|---|---|---|
| | | Catalyst | | | gas flow | acid |
| Experimental | Catalyst | loading | Reaction | Carrier | amount | yield |
| Example | type | amount | temperature | gas | (sccm) | (%) |
| Experimental | Example 1 | 0.4 | 280 | N₂ | 25 | 94.9 |
| Example 1 | (HAP + β- | | | | | |
| | Ca₂P₂O₇, 20:80) | | | | | |
| Experimental | Example 1 | 0.4 | 300 | N₂ | 25 | 92.5 |
| Example 2 | (HAP + β- | | | | | |
| | Ca₂P₂O₇, 20:80) | | | | | |
| Experimental | Example 2 | 0.4 | 280 | N₂ | 25 | 87.3 |
| Example 3 | (HAP + β- | | | | | |
| | Ca₂P₂O₇, 70:30) | | | | | |
| Experimental | Example 3 | 0.4 | 280 | N₂ | 25 | 87.6 |
| Example 4 | (HAP + β- | | | | | |
| | Ca₂P₂O₇, 15:85) | | | | | |
| Experimental | Comparative | 1.6 | 280 | N₂ | 5 | 75.6 |
| Example 5 | Example 1 | | | | | |
| | (HAP) | | | | | |
| Experimental | Comparative | 1.6 | 280 | N₂ + Ar | 50 | 62.8 |
| Example 6 | Example 1 | | | | | |
| | (HAP) | | | | | |
| Experimental | Comparative | 0.4 | 280 | N₂ | 25 | 81.2 |
| Example 7 | Example 2 | | | | | |
| | (β-Ca₂P₂O₇) | | | | | |
| Experimental | Comparative | 0.4 | 280 | N₂ | 25 | 77.3 |
| Example 8 | Example 3 | | | | | |
| | (TiO₂) | | | | | |
| Experimental | Comparative | 0.4 | 280 | N₂ | 25 | 73.4 |
| Example 9 | Example 4 | | | | | |
| | (Silica) gel | | | | | |

Although the titanium dioxide and silica gel catalysts used in Experimental Examples 8 and 9 had been known to have an excellent effect on the conventional 3-hydroxypropionic acid dehydration reaction, it was confirmed from Table 1 above that the yield of acrylic acid was less than 80%, and thus there was a limit to the yield.

Further, it was confirmed from Table 1 above that the yields of acrylic acid were lower when the single-phase catalysts of hydroxyapatite (HAP) were used in Experimental Examples 5 and 6 even when the catalyst loading amounts were increased.

It was confirmed from Experimental Examples 1, 3, 4, and 7 of Table 1 above that the yield of acrylic acid was reduced when the β-calcium pyrophosphate single-phase catalyst was used compared to of the yields when the catalysts having a mixed phase of hydroxyapatite and calcium pyrophosphate was used even when acrylic acid was obtained under the same catalyst conditions.

Meanwhile, even under the same catalyst conditions, it was confirmed that when the catalysts having a mixed phase of hydroxyapatite and calcium pyrophosphate were used, higher yields of acrylic acid were obtained compared to when β-calcium pyrophosphate single-phase catalyst, or TiO₂ or silica gel was used as a catalyst, and it was confirmed that when the catalysts having a mixed phase of hydroxyapatite and calcium pyrophosphate were used, high yields of acrylic acid were obtained even though the catalyst loading amounts were small compared to the single-phase hydroxyapatite catalyst.

That is, it was confirmed that the catalysts having the mixed phase according to the present disclosure has an effect of producing a high acrylic acid yield when used in a process for producing acrylic acid from the dehydration reaction of 3-hydroxypropionic acid.

1. A method for preparing a catalyst for a dehydration reaction of 3-hydroxypropionic acid, the catalyst including hydroxyapatite (HAP) and calcium pyrophosphate, the method comprising the steps of:
dropping a first phosphate solution into a first calcium salt solution to produce an apatite cake;
dropping a second phosphate solution into a second calcium salt solution to produce a calcium pyrophosphate (CaPP) cake;
mixing the apatite cake and the calcium pyrophosphate cake to produce a calcium phosphate cake; and
firing the calcium phosphate cake.

2. The method of claim 1, wherein a weight ratio of the apatite cake and the calcium pyrophosphate cake in the step of mixing the apatite cake and the calcium pyrophosphate cake is 10:90 to 80:20.

3. The method of claim 1, wherein a calcium salt concentration of each of the first calcium salt solution and the second calcium salt solution is 0.1 M or more and 5 M or less.

4. The method of claim 1, wherein a phosphate concentration of each of the first phosphate solution and the second phosphate solution 0.1 M or more and 5 M or less.

5. The method of claim 1, wherein the step of dropping the first phosphate solution into the first calcium salt solution comprises a step of dropping the first phosphate solution into a bath containing the first calcium salt solution, and the step of dropping the second phosphate solution into the second calcium salt solution comprises a step of dropping the second phosphate solution into a bath containing the second calcium salt solution.

6. The method of claim 1, wherein the each of the step of dropping the first phosphate solution into the first calcium salt solution and the step of dropping the second phosphate solution into the second calcium salt solution is are each performed for 30 seconds or more and 1 hour or less.

7. The method of claim 1, wherein the first phosphate solution comprises one or more selected from the group consisting of Li₃PO₄, Na₃PO₄, K₃PO₄, and hydrates thereof.

8. The method of claim 1, wherein the second phosphate solution comprises one or more selected from the group consisting of pyrophosphoric acid (H₄P₂O₇), Li₄P₂O₇, Na₄P₂O₇, and K₄P₂O₇, and hydrates thereof.

9. The method of claim 1, wherein each of the first calcium salt solution and the second calcium salt solution comprises one or more calcium salts selected from the group consisting of calcium chloride, calcium nitrate, calcium sulfate, calcium acetate, and hydrates thereof.

10. The method of claim 1, wherein the step of mixing the apatite cake and the calcium pyrophosphate cake comprises the steps of: preparing a calcium phosphate slurry by mixing the apatite cake and the calcium pyrophosphate cake; and filtering, washing, and drying the calcium phosphate slurry to prepare a calcium phosphate cake.

11. The method of claim 10, wherein the step of preparing the calcium phosphate slurry comprises a step of stirring a mixture of the apatite cake and the calcium pyrophosphate cake at a temperature of 20° C. to 60° C. for 1 hour to 48 hours.

12. The method of claim 1, wherein the firing is performed at a temperature of 300° C. to 700° C.

13. A catalyst for dehydration reaction of 3-hydroxypropionic acid, the catalyst including a hydroxyapatite phase and a calcium pyrophosphate phase, wherein a weight ratio of the hydroxyapatite phase and the calcium pyrophosphate phase is 10:90 to 80:20.

15. A method for producing acrylic acid, the method comprising a step of dehydrating 3-hydroxypropionic acid in the presence of the catalyst according to claim 13.

15. The method of claim 14, wherein the step of dehydrating 3-hydroxypropionic acid is carried out at a temperature of 70° C. to 400° C.
